(19) United States
(12) Patent Application Publication
(10) Pub. No.: US 2022/0064643 A1
(43) Pub. Date: Mar. 3, 2022

(54) Methods for preventing and treating or diagnosing coronavirus infection

(71) Applicants: Peking University, Beijing (CN); Institute of Biophysics, Chinese Academy of Sciences, Beijing (CN); Institute of Laboratory Animal Science, Chinese Academy of Medical Science, Beijing (CN)

(72) Inventors: Fuping YOU, Beijing City (CN); Xiangxi WANG, Beijing City (CN); Chuan QIN, Beijing City (CN)

(21) Appl. No.: 17/461,870

(22) Filed: Aug. 30, 2021

(30) Foreign Application Priority Data
Aug. 31, 2020 (CN) 2020108920106
Nov. 19, 2020 (CN) 2020113015284

(51) Int. Cl. C12N 15/113; A61K 49/00; A61K 31/4704; C07K 16/24; A61P 31/14

(52) U.S. Cl. CPC C12N 15/113; A61K 49/0008; A61P 31/14; C07K 16/24; A61K 31/4704

(57) ABSTRACT

The present disclosure relates to a method for preventing and treating or diagnosing coronavirus infection. SARS-CoV2 lung infection animal models are established using rhesus macaques and human ACE2 transgenic mice, innate immune characteristics that occur in the body in the early stage of SARS-CoV2 infection are explored, and S100A8 protein is found as the root cause initiating the inflammatory storm and becomes an immune indicator for early diagnosis of a patient with SARS-CoV2 infection. Then inhibition of the function of S100A8/S100A9 dimer finally inhibits the virus titer in tissues, thereby decreasing the inflammation level, which proves that a dimer inhibitor can be used as an effective drug for preventing and treating coronavirus infection.

### TECHNICAL FIELD

The present disclosure relates to the field of medicine, in particular to a method for preventing and treating or diagnosing coronavirus infection.

### Background Art

The coronavirus is a single-stranded RNA virus whose main hosts are bats and birds. In recent decades, several coronaviruses have broken through the species boundary between bats and humans, seriously endangering people's life safety and causing great social panics. Severe acute respiratory syndrome caused by SARS-CoV in 2003 eventually led to 8,096 infections and 774 deaths; and in 2012, Middle East respiratory syndrome caused by MERS-CoV eventually led to 2494 infections and 858 deaths. In 2020, large-scale epidemic outbreaks caused by SARS-CoV2 has been found all over the world, becoming the most serious natural disaster in recent 100 years. Epidemics continue to break out in various countries around the world, and the regular epidemic has also caused a great impact on the world economy. Therefore, the prevention and treatment issues of pneumonia caused by the novel coronavirus infection have become common difficulties in the scientific research community.

Generally speaking, anti-virus vaccines are universal means to prevent the viral transmission. However, unlike other viruses, coronaviruses, due to their own particularities, have an ADE (antibody-dependent enhancement) effect on antibodies produced in human bodies after vaccination of several vaccines, comprising inactivated and attenuated live vaccines, so that the antibodies are difficult to protect the body against virus invasion. Likewise, anti-virus antibodies are also the last means to treat virus infection, and antibodies against specific target points of coronaviruses can not eliminate the viruses because of the ADE effect. Therefore, before the problems are solved and effective vaccines are developed, the early diagnosis and symptomatic treatment of the novel coronavirus pneumonia are the top priorities in controlling the epidemic.

Regarding the symptomatic treatment of the novel coronavirus pneumonia, most of the current people's concerns are on the specific innate immune phenotype and inflammatory storm caused by the novel coronavirus. The first is the inflammatory phenotype. When the human body suffers from common virus infections, there will generally be increased lymphocytes and unchanged or reduced neutrophils in the hemogram. In contrast, the coronavirus pneumonia will have changes of hemogram similar to bacterial infection, and patients usually have increased neutrophils and unchanged or decreased lymphocytes. The reason why the bacterial immune phenotype appears in the coronavirus pneumonia is a problem that people have not explained clearly. Then regarding the inflammatory storm., the secretion of inflammatory cytokines and the infiltration of inflammatory cells are important links for the body to repair damage. However, in the coronavirus pneumonia, patients usually eventually develop an uncontrolled high-intensity inflammatory cytokine storm, and such uncontrolled inflammation not only does not facilitate the repair of damaged lung tissue, but is a major factor for the fatal multiple organ failure in the late stage of coronavirus pneumonia. The initial secretion of a small number of inflammatory cytokines recruits inflammatory cells to perform infiltration and be activated, then the activated inflammatory cells further secrete inflammatory factors, so that the signal is amplified step by step, resulting in the inflammatory storm. Therefore, the inflammation can be suppressed most effectively and symptomatic treatment can be carried out, by studying the early coronavirus pneumonia and discovering the root cause of the inflammatory storm. However, patients with coronavirus pneumonia have a long incubation period, for example, SARS-CoV2 has an incubation period of about 7 to 14 days. Therefore, scientific researchers can not find the coronavirus pneumonia patients in the early stage to study the root cause of inflammation. The currently known research results show that, unlike normal virus infections, SARS-CoV, MERS-CoV and SARS-CoV2 do not cause the normal expression of antiviral cytokine interferons in the early stage of infection. This may be one of the reasons for the inflammation staying at a high level, but it does not reveal the occurrence of inflammatory storm and specific treatment can not be achieved.

The long incubation period of SARS-CoV2 is the root cause that the virus infection can not be diagnosed early. The diagnosis of virus infection is usually via a nucleic acid detection; however, for the patients infected with SARS-CoV2, the virus nucleic acid can not be detected during the incubation period. At the same time, patients in the incubation period also have the ability to transmit, which creates a difficulty in controlling the transmission of the epidemic.

### SUMMARY OF THE INVENTION

In view of the technical problems existing in the prior art, the inventors use rhesus macaques and human ACE2 transgenic mice to establish SARS-CoV2 lung infection animal models. Innate immune characteristics that occur in the body in the early stage of SARS-CoV2 infection are explored, and S100A8 protein is found as the root cause initiating the inflammatory storm and becomes an immune indicator for early diagnosis of a patient with SARS-CoV2 infection. Then inhibition of the function of S100A8/S100A9 protein by drug paquinimod finally inhibits the virus titer in tissues, thereby decreasing the inflammation level, and effectively suppressing the development and progression of SARS-CoV2 pneumonia in the animal model. Specifically, the present disclosure comprises the following contents.

The first aspect of the present disclosure provides a method for preventing and treating a disease caused by a coronavirus, comprising administering a S100A8/S100A9 dimer activity inhibitor to a subject in need thereof.

In certain embodiments, the S100A8/S100A9 dimer activity inhibitor is a small molecule compound having a structure represented by the following Formula (I) or (II):

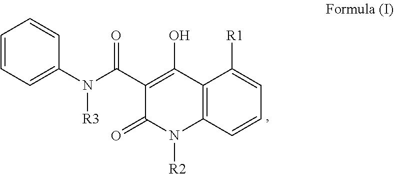

wherein each of R1 to R3 independently represents C1-C5 alkyl;

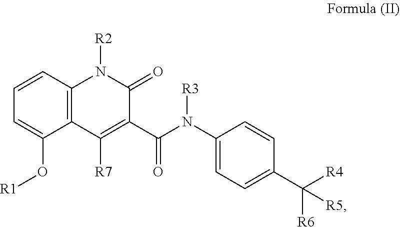

wherein each of R1 to R3 independently represents C1-C5 alkyl, each of R4 to R6 independently represents a halogen atom, and R7 represents hydroxyl or a hydrogen atom.

In certain embodiments, the S100A8/S100A9 dimer activity inhibitor is paquinimod and/or tasquinimod.

In certain embodiments, the S100A8/S100A9 dimer activity inhibitor is a shRNA that targets S100A8 gene or S100A9 gene.

In certain embodiments, the S100A8/S100A9 dimer activity inhibitor is an antibody against S100A8 or a fragment thereof and/or an antibody against S100A9 or a fragment thereof.

The second aspect of the present disclosure provides a method for diagnosing coronavirus infection, comprising detecting the amount of S100A8 protein or its gene expression level, the amount of S100A9 protein or its gene expression level, or the amount of S100A8/S100A9 dimer.

In certain embodiments, primers and/or probes are used to detect the expression level of S100A8 gene or S100A9 gene.

In certain embodiments, the amount of S100A8 protein, S100A9 protein, or S100A8/S100A9 dimer is detected using an antibody against S100A8 or a fragment thereof, or an antibody against S100A9 or a fragment thereof.

The third aspect of the present disclosure provides a method for screening a compound useful for preventing and treating or alleviating coronavirus infection, comprising measuring the expression level of S100A8 gene and/or S100A9 gene, or the amount of S100A8 and/or S100A9 in a sample.

In certain embodiments, the method of the third aspect comprises:

a. measuring the expression level of S100A8 gene and/or S100A9 gene, or the amount of S100A8 and/or S100A9 in a biological sample collected from a non-human subject infected with a coronavirus, to obtain a first measurement value;

b. administering a test compound to the non-human subject;

c. measuring the expression level of S100A8 gene and/or S100A9 gene, or the amount of S100A8 and/or S100A9 in a biological sample collected from the non-human subject after administration of the test compound, to obtain a second measurement value;

d. comparing the first measurement value and the second measurement value; and

e. if the second measurement value is less than the first measurement value, the test compound is screened as a compound useful for preventing and treating or alleviating coronavirus infection, and if the second measurement value is greater than or equal to the first measurement value, the test compound is screened as a compound useless for preventing and treating or alleviating coronavirus infection;

alternatively, the method comprises:

a. measuring the expression level of S100A8 and/or S100A9 in a cell overexpressing S100A8 and/or S100A9 to obtain a first measurement value;

b. administering a test compound to the cell;

c. measuring the expression level of S100A8 gene and/or S100A9 gene in the cell after administration of the test compound to obtain a second measurement value;

d. comparing the first measurement value and the second measurement value; and

e. if the second measurement value is less than the first measurement value, the test compound is screened as a compound useful for treating or alleviating coronavirus infection, and if the second measurement value is greater than or equal to the first measurement value, the test compound is screened as a compound useless for treating or alleviating coronavirus infection.

In certain embodiments, the non-human subject in the method of the third aspect is an animal model with manifestations of severe pneumonia.

In certain embodiments, the animal model has manifestations of severe pneumonia by infection with MHV, wherein the animal model is an IFNAR gene knockout animal

In certain embodiments, the method of the third aspect further detecting immature neutrophils in the animal model.

In certain embodiments, the animal comprises mice, ferrets, and monkeys.

In certain embodiments, the coronavirus comprises SARS-CoV-2 and/or MHV-A59.

In certain embodiments, the method of the third aspect comprises:

(1) making an animal model have manifestations of severe pneumonia by infection with MHV;

(2) administering a test compound to the animal model with manifestations of severe pneumonia; and

(3) if the manifestations of severe pneumonia of the animal model are relieved, remitted or eliminated, the test compound is identified as a compound useful for preventing, treating or alleviating the coronavirus, otherwise the test compound is identified as a compound not useful for preventing, treating or alleviating the coronavirus;

alternatively, the method comprises:

(1) infecting an animal model with MHV, and then detecting the proliferation or activation of immature neutrophils in vivo in the animal model, wherein the animal model is an IFNAR gene knockout animal;

(2) administering a test compound to the animal model after infection, and then detecting the proliferation or activation of immature neutrophils in the animal model; and

(3) if the proliferation or activation of immature neutrophils in (2) is inhibited or slowed down, the test compound is identified as a compound useful for preventing, treating or alleviating the coronavirus, otherwise the test compound is identified as a compound not useful for preventing, treating or alleviating the coronavirus.

The fourth aspect of the present disclosure provides a method for pathological state and drug evaluation for the novel coronavirus pneumonia, comprising using an IFNAR gene knockout animal model with manifestations of severe pneumonia, or comprising detecting abnormal proliferation of immature neutrophils in the animal model.

The fifth aspect of the present disclosure provides an animal model, which is an IFNAR gene knockout animal model with manifestations of severe pneumonia.

### BRIEF DESCRIPTION OF THE DRAWINGS

FIG. 1 shows the results of clustering analysis of transcriptional activity in the lung in the early stage of SARS-CoV2 infection.

FIG. 2 shows the results of clustering analysis of the increase in the number of neutrophils and the chemotaxis of inflammatory storm.

FIG. 3 shows the analysis results of alarmin protein based on the S100A8 gene.

FIG. 4 shows the results of transcript analysis on lung tissues of patients with SARS-CoV2 pneumonia.

FIG. 5 shows the results of transcript analysis on lung tissues of human ACE2 transgenic mice infected with SARS-CoV2.

FIG. 6 shows the results of S100A8 gene expression in IFNar gene knockout mice infected with MHV.

FIG. 7 shows the results of S100A8 gene expression in IFNar knockout mice and human ACE2 mice infected with the respiratory virus IAV.

FIG. 8 shows the results of differential expression analysis based on the whole genome.

FIG. 9 shows that IFNAR gene-deficient mice died of SARS-CoV-2 and MHV-A59 infections. A. Wild-type and IFNAR gene-deficient mice were infected with MHV-A59 (100 pfu/mouse) by means of nasal drops. B. A. Wild-type and IFNAR gene-deficient mice were first infected with the lentivirus packaging ACE2, and then infected with SARS-CoV-2 (100 pfu/mouse) by means of nasal drops.

FIG. 10 shows that AEC2 humanized mice will not cause severe illness after SARS-CoV-2 infection. AEC2 humanized mice were infected with SARS-CoV-2 (100 pfu/mouse) by means of nasal drops.

FIG. 11 shows that SARS-CoV-2 and MHV-A59 infections caused severe lung injury in IFNAR gene-deficient mice. Wild-type and IFNAR gene-deficient mice were infected with MHV-A59 (100 pfu/mouse) by means of nasal drops. Wild-type and IFNAR gene-deficient mice were first infected with the lentivirus packaging ACE2, and then infected with SARS-CoV-2 (100 pfu/mouse) by means of nasal drops. Then the lungs were taken for sectioning and HE staining.

FIG. 12 shows that SARS-CoV-2 and MHV-A59 infections caused similar immune responses in IFNAR gene-deficient mice. Wild-type and IFNAR gene-deficient mice were infected with MHV-A59 (100 pfu/mouse) by means of nasal drops. Wild-type and IFNAR gene-deficient mice were first infected with the lentivirus packaging ACE2, and then infected with SARS-CoV-2 (100 pfu/mouse) by means of nasal drops. Then the lung tissues were taken for RNAseq, and functional clustering analysis was performed on the genes that were induced for up-regulated expression.

FIG. 13 shows abnormal activation of neutrophils in IFNAR gene-deficient mice infected with SARS-CoV-2 and MHV-A59. Wild-type and IFNAR gene-deficient mice were infected with MHV-A59, IAV, EMCV, HSV-1 and LPS (100 pfu/mouse) by means of nasal drops or treated (LPS). Wild-type and IFNAR gene-deficient mice were first infected with the lentivirus packaging ACE2, and then infected with SARS-CoV-2 (100 pfu/mouse) by means of nasal drops. Then the blood was taken for flow cytometry analysis (antibodies were against LY6G and CD11b).

FIG. 14 shows that drug treatments inhibited abnormal activation of neutrophils in IFNAR gene-deficient mice infected with SARS-CoV-2. Wild-type and IFNAR gene-deficient mice were first infected with the lentivirus packaging ACE2, and then infected with SARS-CoV-2 (100 pfu/mouse) by means of nasal drops. Then the blood was taken for flow cytometry analysis (antibodies were against LY6G and CD11b).

FIG. 15 shows that drug treatments inhibited abnormal activation of neutrophils in IFNAR gene-deficient mice infected with MHV-A59. Wild-type and IFNAR gene-deficient mice were infected with MHV-A59 (100 pfu/mouse) by means of nasal drops. Then the blood was taken for flow cytometry analysis (antibodies were against LY6G and CD11b).

FIG. 16 shows that wild-type and IFNAR gene-deficient mice were infected with MHV-A59 (100 pfu/mouse) by means of nasal drops.

FIG. 17 shows the specific experimental results of the resistance of drug paquinimod to coronavirus infection.

FIG. 18 shows the results of drug inhibition of MHV infection of lung tissues in IFNar knockout mice.

FIG. 19 shows graphs showing the results of the expression levels of S100A8/S100A9 in the lung which were reduced using shRNAs against S100A8 and S100A9.

### DETAILED DESCRIPTION OF EMBODIMENTS

Various exemplary implementations of the present disclosure are now described in detail. The detailed description should not be considered as a limitation on the present disclosure, but should be understood as a more detailed description of certain aspects, characteristics, and embodiments of the present disclosure.

It should be understood that the terms described in the present disclosure are only used to describe specific implementations, rather than to limit the present disclosure. In addition, for the numerical ranges in the present disclosure, it should be understood that the upper limit and the lower limit of the range and each intermediate value between them are specifically disclosed. Each smaller range between an intermediate value among any stated values or within any stated range and an intermediate value among any other stated values or within any other stated range is also encompassed in the present disclosure. The upper and lower limits of these smaller ranges can be independently included or excluded from the range.

Unless otherwise specified, all technical and scientific terms used herein have the same meaning as commonly understood by those of ordinary skill in the art to which the present disclosure belongs. Although the present disclosure only describes preferred methods and materials, any methods and materials similar or equivalent to those described herein can also be used in the implementation or testing of the present disclosure. All documents mentioned in this specification are incorporated by reference to disclose and describe methods and/or materials related to the documents. In the event of conflict with any incorporated document, the content of this specification shall prevail. Unless otherwise specified, “%” or “amount” is a percentage based on weight.

### EXAMPLE 1

I. S100A8/S100A9 are Effective Target Points for the Treatment of Novel Coronavirus-Related Pneumonia

Using rhesus macaques and human ACE2 transgenic mice, animal models of SARS-CoV2 lung infection were established. Innate immune characteristics that occur in the body in the early stage of SARS-CoV2 infection are explored, and S100A8 protein is found as the root cause initiating the inflammatory storm and becomes an immune indicator for early diagnosis of a patient with SARS-CoV2 infection. Then inhibition of the function of S100A8/S100A9 protein by drug paquinimod finally inhibits the virus titer in tissues, thereby decreasing the inflammation level, and effectively suppressing the development and progression of SARS-CoV2 pneumonia in the animal model.

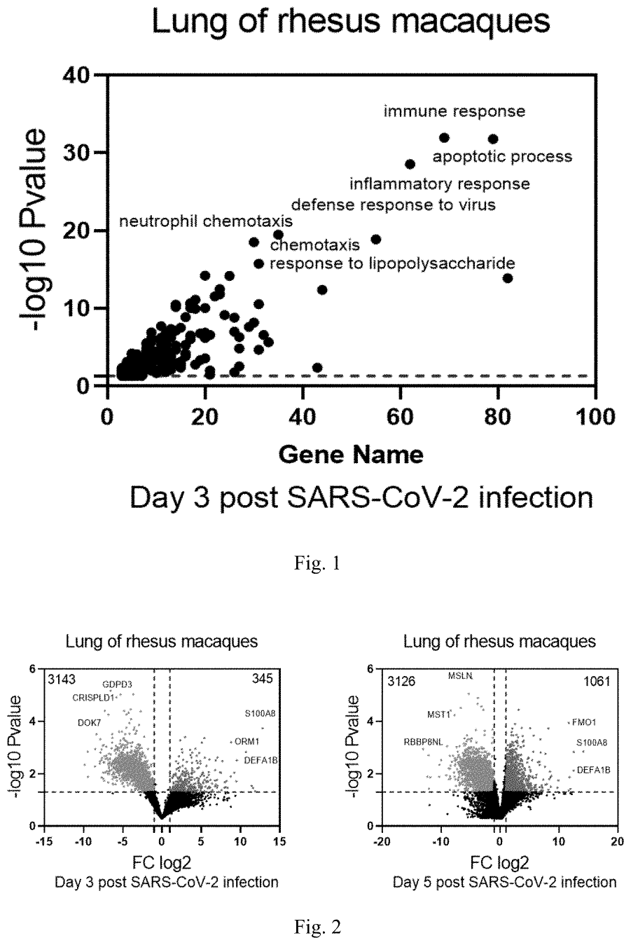

First, rhesus macaques were infected with SARS-CoV2 through nasal drops, and the lung tissues on days 0, 3, and 5 were taken for RNA-seq sequencing to analyze the changes of transcriptional activity in the lung in the early stage of SARS-CoV2 infection. As shown in the results of FIG. 1, it was found through GO analysis that the up-regulated genes were mainly clustered in the directions of immunity, inflammation, and neutrophil chemotaxis. It was revealed that in the early stage of SARS-CoV2 infection, the lungs of rhesus macaques had already showed inflammatory storm tendency and neutrophil infiltration and activation, which proved the successful establishment of the rhesus macaque animal model of SARS-CoV2 infection, and also suggested that the increase of neutrophils in the clinical symptoms in SARS-CoV2 infected patients was an early pathological event.

By comparing the genes in the neutrophil chemotaxis clustering, the reason was elucidated for that SARS-CoV2 pneumonia was different from common viruses causing the increase in the number of neutrophils and the inflammatory storm. The comparison results were shown in FIG. 2. Among all the up-regulated genes, the S100A8 gene was the most obvious, and it played an important role in neutrophil chemotaxis.

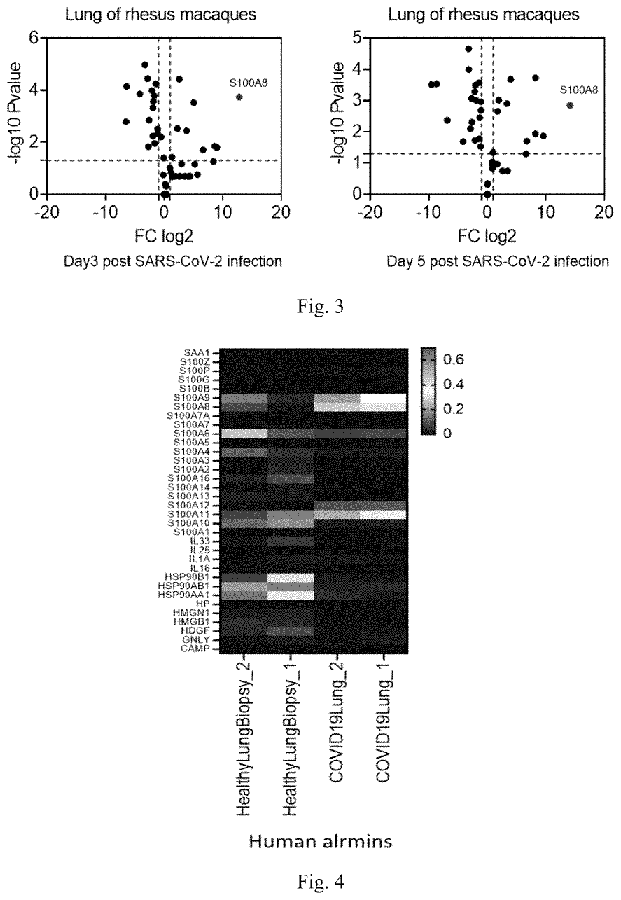

S100A8 protein is a classic alarmin protein. Stimulation of cells of the body by external stress (such as activation of the DAMP signaling pathway) or tissue damage would lead to the transcription and secretion of alarmins, thereby recruiting inflammatory cells to perform infiltration and mediate inflammation response. Through the analysis of known alarmins, with the analysis results shown in FIG. 3, it can be found that in the early stage of SARS-CoV2 infection, the expression of alarmins in the rhesus macaque lung tissues was not extensively up-regulated, and the up-regulation of S100A8 gene expression was extremely specific in the early stage of SARS-CoV2 infection. It was suggested that S100A8 protein was an important part leading to the inflammatory storm in the pathogenic process of SARS-CoV2 pneumonia.

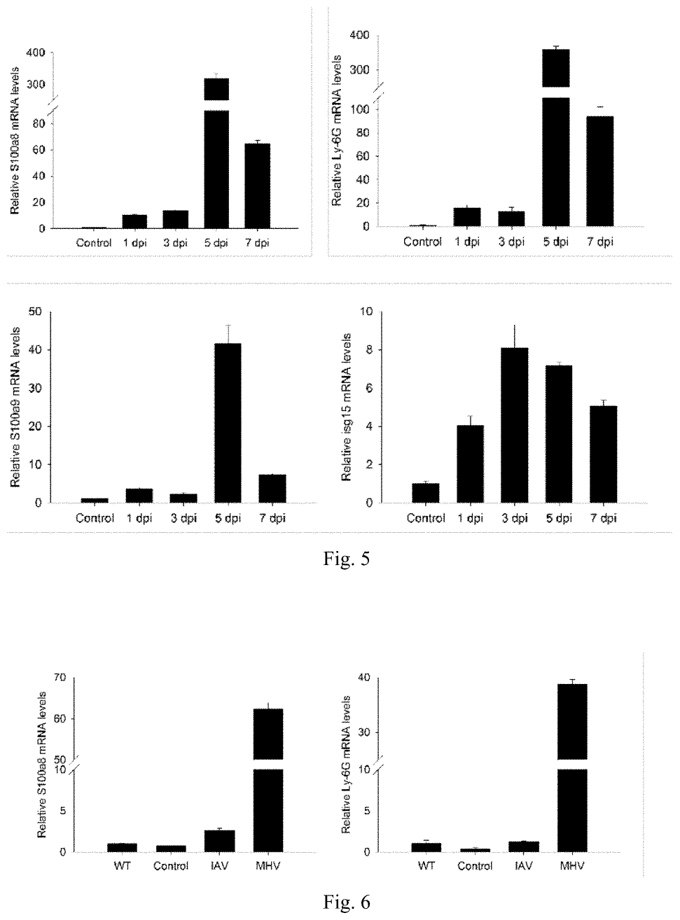

In order to further confirm that the up-regulation of S100A8 gene expression is an important sign of SARS-CoV2 pneumonia, we also performed transcript analysis on both lung tissues of SARS-CoV2 pneumonia patients and lung tissues of human ACE2 transgenic mice infected with SARS-CoV2. The results of transcript analysis on lung tissues of SARS-CoV2 pneumonia patients were shown in FIG. 4, and the results of transcript analysis on lung tissues of human ACE2 transgenic mice infected with SARS-CoV2 were shown in FIG. 5. The results showed that both the lung tissues of SARS-CoV2 pneumonia patients and human ACE2 transgenic mice infected with SARS-CoV2 showed up-regulation of S100A8 gene expression.

The up-regulation of S100A8 gene expression in the patients' lung tissue samples was not very strong, possibly because the samples were in the late stage of infection. The mouse samples showed that expression of S100A8 gene had been up-regulated on the first day of infection with addition of viruses and reached the peak on the fifth day. It was proved that the up-regulation of S100A8 gene expression was initiated in the early stage of infection, and was at a position close to the beginning during the amplification of the inflammatory storm. The up-regulation trend of S100A8 gene expression revealed the role of S100A8 protein in mediating neutrophil infiltration and activation in SARS-CoV2 pneumonia. The above results showed that the up-regulation of S100A8 protein occurs in the early stage of SARS-CoV2 pneumonia, and the up-regulation of specific gene expression was one of the key reasons for the recruitment of neutrophils in SARS-CoV2 infection.

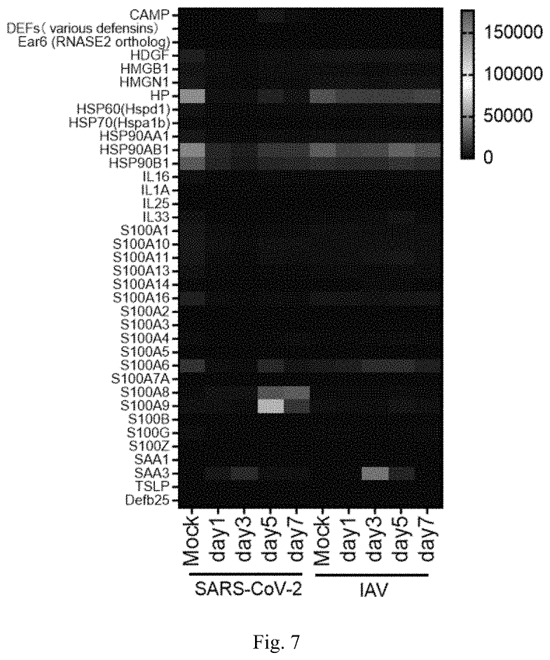

Experiments were conducted on mouse animal models infected with different viruses to explore whether the up-regulation of S100A8 was SARS-CoV2 specific or universal. The first was to study another coronavirus MHV that used mice as the host. The experimental results were shown in FIG. 6 that infection with MHV in IFNar gene knockout mice led to the up-regulation of S100A8 gene expression. The experimental results of IAV, another respiratory virus that used mice as the host, were shown in FIG. 6 and FIG. 7, and infection with respiratory virus IAV in IFNar knockout mice and human ACE2 mice did not cause up-regulation of S100A8. This proved that the up-regulation of S100A8 gene expression was specific to coronavirus, revealing the possibility of S100A8 serving as an immunological indicator for early diagnosis of coronavirus infection.

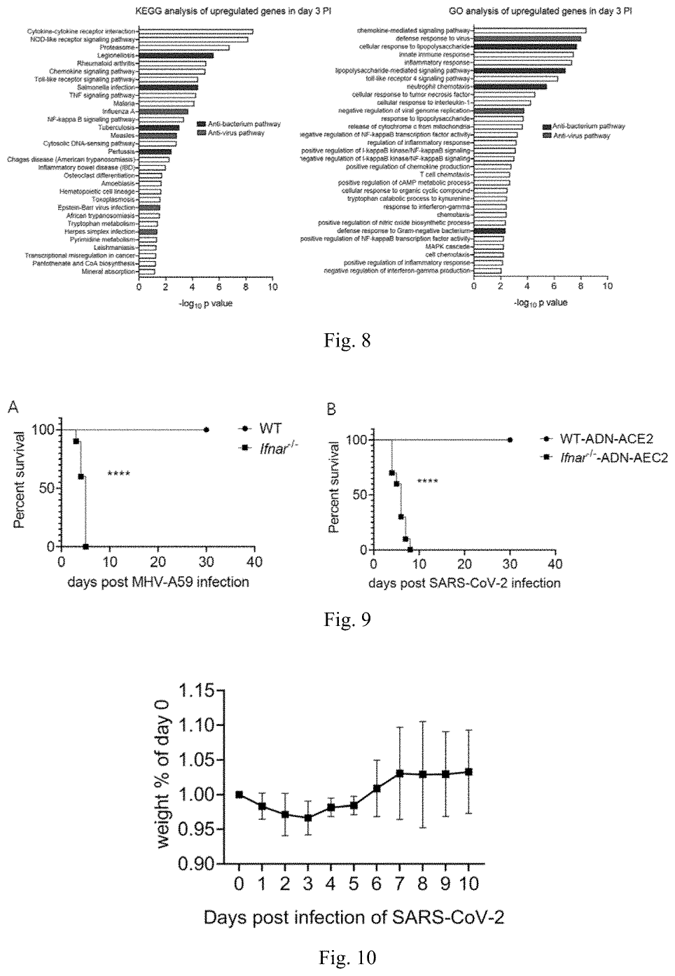

Under normal physiological conditions, the background expression level of S100A8 protein was not high, and S100A9 protein was normally expressed and localized in the cytoplasm. In the case of signal stimulation, S100A8 protein was expressed in large quantities, and formed a dimer with S100A9 protein in the cytoplasm, the dimer was secreted to the outside of the cell to recruit neutrophils and bind to the membrane receptor TLR4, thereby activating downstream inflammation signaling to initiate inflammation response. TLR4 was a well-known pattern recognition receptor (PRR). A large number of studies had shown that its main function was to recognize lipopolysaccharide (LPS) as the Grain-negative bacterial pathogen-associated molecular pattern (PAMP), thereby activating downstream signaling pathways to initiate innate immunity and inflammation response. The specific up-regulation of S100A8 protein in coronavirus infection activated the TLR4 receptor, as shown in FIG. 8, which explained why coronavirus pneumonia was different from common virus infections in which a rise of lymphocytes and a decline of neutrophils would occur, but instead there would be hemogram similar to bacterial infection with increased neutrophils and decreased lymphocytes.

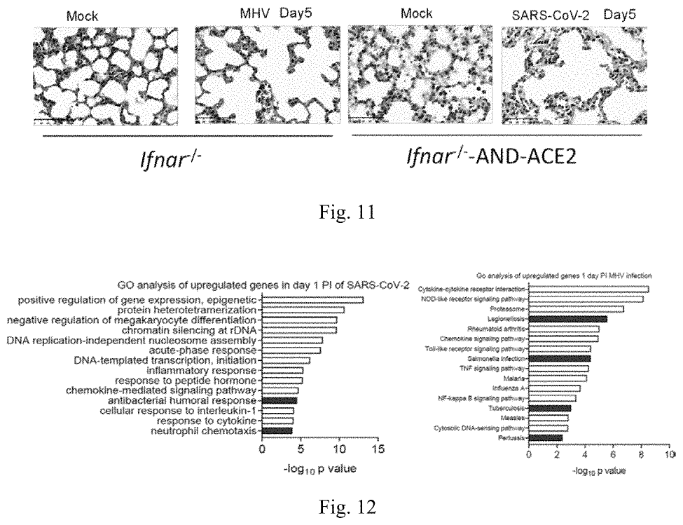

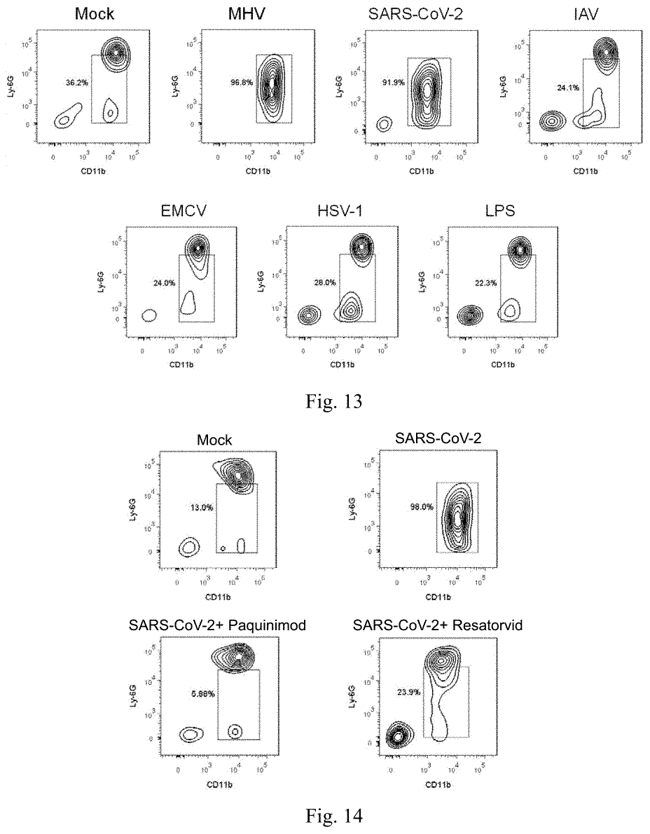

Next, the inventors conducted a systematic screening of mice with edited genes related to inflammation response and found that IFNAR gene-deficient mice exhibited significant phenotypes of severe pneumonia after been infected with novel coronavirus (SARS-CoV-2) and mouse coronavirus (MHV-A59), and all died within 10 days (FIG. 9). Furthermore, ACE2 humanized mice infected with SARS-CoV-2 would lose weight. However, the weight would fully recover in about 7 days and severe illness and death of mice did not appear (FIG. 10). Lung sections of mice showed that IFNAR knockout mice had severe lung damage (FIG. 11). By extracting RNAs from lungs of infected mice, performing a genome-wide RNA-seq analysis, and further performing GO functional analysis of the differentially expressed genes, it was found that both of the immune responses of lungs of IFNAR mice after infection with MHV and SARS-CoV-2 were manifested by a significant enhancement of anti-bacterial immune response (FIG. 12), consistent with clinical manifestations (4). The analysis of mouse blood immune cells further confirmed this point. MHV and SARS-CoV-2 infections can cause abnormal proliferation of immature neutrophils, which was consistent with the results of abnormal proliferation of immature neutrophils in severe patients (4). At the same time, during the course of infection with other viruses, no abnormal proliferation of immature neutrophils was found (FIG. 13). It was suggested that the proliferation of immature neutrophils was a unique feature caused by coronavirus infection and can be used as an important evaluation index for the degree of coronavirus infection and the degree of disease progression. Important MHV infection, like SARS-CoV-2 infection, can also cause abnormal proliferation of immature neutrophils. Therefore, after IFNAR mice were infected with MHV, a sharp increase of the immature neutrophils appeared, which can be used as an important model and indicator for evaluating the effectiveness of related drugs and pathological changes under the condition of safety level 2.

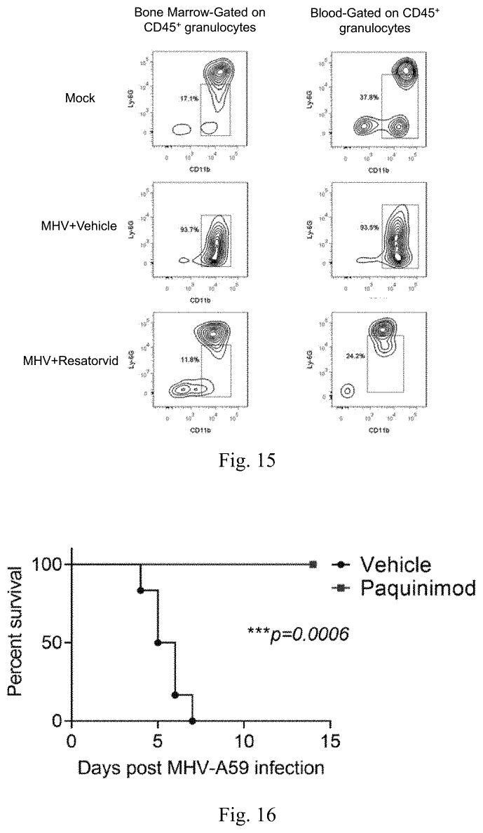

Next, the inventors used small molecule drugs targeting S100A8/A9 and TLR4 to treat mice infected with SARS-CoV-2 and MHV. Drug treatment can remarkably inhibit the proliferation of immature neutrophils caused by SARS-CoV-2 and MHV infections (FIG. 14 and FIG. 15), and can rescue the dying mice caused by MHV infection (FIG. 16). It was further indicated that IFNAR knockout mice infected with MHV can be used as an effective model for the pathological state and drug evaluation for the novel coronavirus pneumonia.

II. Analysis of Potential Drugs for SRAS-CoV2

1. S100A8/S100A9 Dimer Small Molecule Inhibitor

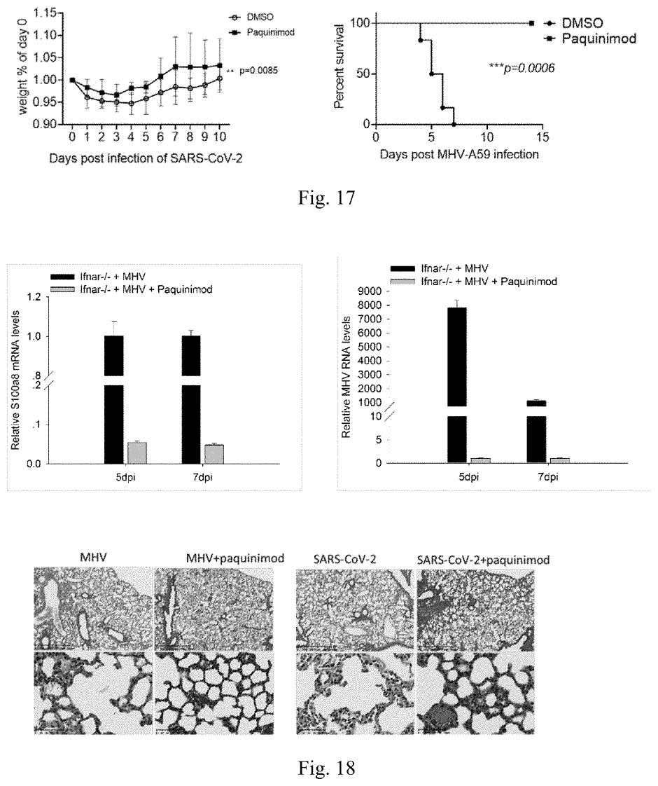

The drug paquinimod was used and studied in the experiment. Paquinimod was a small molecule inhibitor that can effectively inhibit the binding of S100A8/S100A9 dimer to TLR4, thereby inhibiting activation of downstream inflammation signaling. The results were shown in FIG. 17. The results showed that compared with the control group, human ACE2 mice in the paquinimod nasal drop group were significantly more resistant to SRAS-CoV2; IFNar knockout mice infected with MHV in the paquinimod nasal drop group exhibited an increased survival rate; while there was no significant difference in the survival rate of IAV-infected mice in the paquinimod nasal drop group. It was suggested that paquinimod can specifically improve the resistance to coronavirus infection.

Subsequent analysis of MHV-infected lung tissues of IFNar knockout mice was carried out, and the experimental results were shown in FIG. 18. The results showed that the drug inhibited the activation of TLR4 by the S100A8/S100A9 dimer, so that inflammatory cells were not effectively recruited to aggregate and subsequent lung S100A8 gene expression was not up-regulated, thereby reducing inflammation and lung damage caused by inflammation, and inhibiting replication of SARS-CoV-2 and MHV. The above results proved that paquinimod can inhibit the activation of neutrophils caused by coronavirus infection by inhibiting the function of S100A8 protein, and strangle the uncontrolled inflammatory storm in the cradle. Therefore, paquinimod had become a potential drug for symptomatic treatment for SARS-CoV2 pneumonia.

2. shRNAs Targeting S100A8 and S100A9

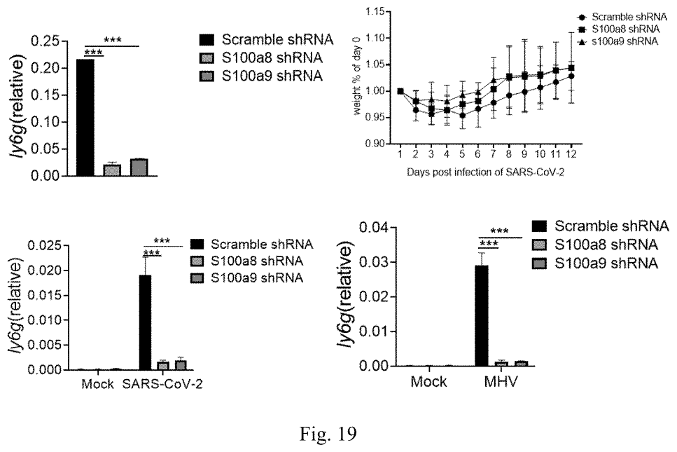

In order to further confirm the immunomodulatory function of S100A8/S100A9 in the novel coronavirus infection, the inventors constructed shRNAs for S100A8 and S100A9 and packaged them into lentivirus. Mice were nasally fed with the lentivirus to reduce the expression level of S100A8/S100A9 in the lungs (as shown in FIG. 19). Moreover, after shRNA knocked down S100A8/S100A9, the weight loss of mice caused by SARS-CoV-2 infection was obviously improved. In terms of molecular mechanism, S100A8/S100A9 shRNAs significantly down-regulated the expression level of LY6G, indicating that the gene silencing technology to target S100A8/S100A9 can also reduce immune cell infiltration and inflammation response caused by SARS-CoV-2.

1. A method for preventing and treating a disease caused by a coronavirus in a subject in need thereof comprising administering to the subject an effective amount of an S100A8/S100A9 dimer activity inhibitor.

2. The method according to claim 1, wherein the S100A8/S100A9 dimer activity inhibitor is a small molecule compound having a structure represented by the following Formula (I) or (II): 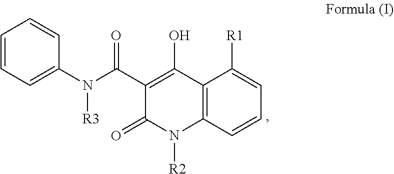
wherein each of R1 to R3 independently represents C1-C5 alkyl; 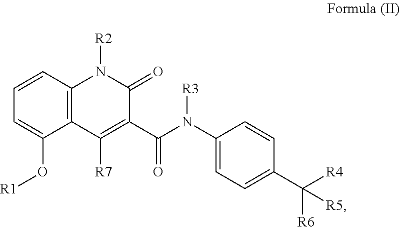
wherein each of R1 to R3 independently represents C1-C5 alkyl, each of R4 to R6 independently represents a halogen atom, and R7 represents hydroxyl or a hydrogen atom.

3. The method according to claim 2, wherein the S100A8/S100A9 dimer activity inhibitor is paquinimod and/or tasquinimod.

4. The method according to claim 1, wherein the S100A8/S100A9 dimer activity inhibitor is a nucleic acid molecule capable of inhibiting the production of S100A8 or S100A9 protein, preferably at least one selected from the group consisting of antisense oligonucleotides, siRNAs, miRNAs and sgRNAs.

5. The method according to claim 1, wherein the S100A8/S100A9 dimer activity inhibitor is an antibody against S100A8 or a fragment thereof, and/or an antibody against S100A9 or a fragment thereof.

6. A method for diagnosing coronavirus infection, comprising detecting the amount of S100A8 protein or its gene expression level, the amount of S100A9 protein or its gene expression level, or the amount of S100A8/S100A9 dimer.

7. The method according to claim 6, wherein primers and/or probes are used to detect the expression level of S100A8 gene or S100A9 gene.

8. The method according to claim 6, wherein the amount of S100A8 protein, S100A9 protein or S100A8/S100A9 dimer is detected using an antibody against S100A8 or a fragment thereof, or an antibody against S100A9 or a fragment thereof.

9. A method for screening a compound useful for preventing and treating or alleviating coronavirus infection, comprising measuring the expression level of S100A8 gene and/or S100A9 gene, or the amount of S100A8 and/or S100A9 in a sample.

10. The method according to claim 9, wherein the method comprises:
a. measuring the expression level of S100A8 gene and/or S100A9 gene, or the amount of S100A8 and/or S100A9 in a biological sample collected from a non-human subject infected with a coronavirus, to obtain a first measurement value;
b. administering a test compound to the non-human subject;
c. measuring the expression level of S100A8 gene and/or S100A9 gene, or the amount of S100A8 and/or S100A9 in a biological sample collected from the non-human subject after administration of the test compound, to obtain a second measurement value;
d. comparing the first measurement value and the second measurement value; and
e. if the second measurement value is less than the first measurement value, the test compound is screened as a compound useful for preventing and treating or alleviating coronavirus infection, and if the second measurement value is greater than or equal to the first measurement value, the test compound is screened as a compound useless for preventing and treating or alleviating coronavirus infection;
alternatively, the method comprises:
a. measuring the expression level of S100A8 and/or S100A9 in a cell overexpressing S100A8 and/or S100A9 to obtain a first measurement value;
b. administering a test compound to the cell;
c. measuring the expression level of S100A8 gene and/or S100A9 gene in the cell after administration of the test compound to obtain a second measurement value;
d. comparing the first measurement value and the second measurement value; and
e. if the second measurement value is less than the first measurement value, the test compound is screened as a compound useful for treating or alleviating coronavirus infection, and if the second measurement value is greater than or equal to the first measurement value, the test compound is screened as a compound useless for treating or alleviating coronavirus infection.

11. The method according to claim 10, wherein the non-human subject is an animal model with manifestations of severe pneumonia.

12. The method according to claim 10, wherein the animal model has manifestations of severe pneumonia by infection with MHV, and wherein the animal model is an IFNAR gene knockout animal

13. The method according to claim 11, further comprising detecting immature neutrophils in the animal model.

14. The method according to claim 11, wherein the animal comprises mice, ferrets, and monkeys.

15. The method according to claim 11, wherein the coronavirus comprises SARS-CoV-2 and/or MHV-A59.

16. The method according to claim 11, wherein the method comprises:
(1) making an animal model have manifestations of severe pneumonia by infection with MHV;
(2) administering a test compound to the animal model with manifestations of severe pneumonia; and
(3) if the manifestations of severe pneumonia of the animal model are relieved, remitted or eliminated, the test compound is identified as a compound useful for preventing, treating or alleviating the coronavirus, otherwise the test compound is identified as a compound not useful for preventing, treating or alleviating the coronavirus;
alternatively, the method comprises:
(1) infecting an animal model with MHV, and then detecting the proliferation or activation of immature neutrophils in vivo in the animal model, wherein the animal model is an IFNAR gene knockout animal;
(2) administering a test compound to the animal model after infection, and then detecting the proliferation or activation of immature neutrophils in the animal model; and
(3) if the proliferation or activation of immature neutrophils in (2) is inhibited or slowed down, the test compound is identified as a compound useful for preventing, treating or alleviating the coronavirus, otherwise the test compound is identified as a compound not useful for preventing, treating or alleviating the coronavirus.

17. A method for pathological state and drug evaluation for the novel coronavirus pneumonia, comprising using an IFNAR gene knockout animal model with manifestations of severe pneumonia, or comprising detecting abnormal proliferation of immature neutrophils in the animal model.

18. An animal model, which is an IFNAR gene knockout animal model with manifestations of severe pneumonia.
